(19) United States
(12) Patent Application Publication
(10) Pub. No.: US 2026/0347306 A1
(43) Pub. Date: Nov. 5, 2026

(54) PROCESSES FOR PRODUCING DIALKYL CARBONATES AND GLYCOLIC COMPOUNDS

(71) Applicant: Lyondell Chemical Technology, L.P., Houston, TX (US)

(72) Inventors: David S. Mannel, Humble, TX (US); Sandor Nagy, Seabrook, TX (US); Ha H. Nguyen, Houston, TX (US); Larry W. Arndt, Humble, TX (US); Amy D. Rose, Humble, TX (US); Barbara Kimmich, Houston, TX (US)

(73) Assignee: Lyondell Chemical Technology, L.P., Houston, TX (US)

(21) Appl. No.: 19/653,805

(22) Filed: Apr. 21, 2026

Related U.S. Application Data
(60) Provisional application No. 63/793,570, filed on Apr. 23, 2025.
(60) Provisional application No. 63/793,572, filed on Apr. 23, 2025.

(51) Int. Cl. C07C 69/96; C07C 31/20; C07C 68/06; C07C 68/08; C07D 301/03; C07D 303/04; C07D 317/36

(52) U.S. Cl. CPC C07C 69/96; C07C 31/20; C07C 68/06; C07C 68/08; C07D 301/03; C07D 303/04; C07D 317/36

(57) ABSTRACT

A process for producing dialkyl carbonates and glycolic compounds. A process may include: (a) epoxidizing an olefin in the presence of a first catalyst to produce a first product stream comprising an alkylene oxide; (b) reacting at least a portion of the first product stream with carbon dioxide in the presence of a second catalyst to produce a second product stream comprising a cyclic carbonate, wherein the second product stream has a water content less than about 0.5 wt % based on the second product stream; and (c) reacting at least a portion of the second product stream with an alcohol in the presence of a third catalyst to produce a third product stream comprising aa dialkyl carbonate.

### PRIOR RELATED APPLICATIONS

This application claims the benefit of priority to U.S. Provisional Application No. 63/793,570, filed on Apr. 23, 2025, and to U.S. Provisional Application No. 63/793,572, filed on Apr. 23, 2025, each of which is incorporated herein by reference in their entirety.

### TECHNICAL FIELD

The present disclosure relates to the production of dialkyl carbonates and glycolic compounds.

### INTRODUCTION

Dialkyl carbonates and compounds/materials derived therefrom have been widely implemented within commercial products and processes, such as chemical synthesis, pharmaceuticals, plastics, lubricants, solvents, plasticizers, and monomers. Given such wide commercial implementation of dialkyl carbonates, various methods and processes have been developed to produce such compounds.

One such method of dialkyl carbonate manufacture includes transesterification of cyclic carbonates, typically involving reacting a cyclic carbonate with an amount of an aliphatic monohydric alcohol in the presence of a catalyst. However, the transesterification process also produces a glycolic compound, which can cause unintended side reactions and/or polymerizations to occur thus reducing dialkyl carbonate yield and purity. In addition, the source of the cyclic carbonate can further introduce additional contaminants that can have deleterious effects on the transesterification reaction.

Common methods for cyclic carbonate formation include the formation of an alkylene oxide and the reaction of such with carbon dioxide while in the presence of a catalyst. As such, an alkylene oxide may be formed via an epoxidation reaction between an olefin and an oxidizing agent in the presence of a catalyst, similar to commercial processes for the production of various glycolic compounds. For example, the production of propylene oxide from propylene and an organic hydroperoxide oxidizing agent, such as ethylbenzene hydroperoxide or tert-butyl hydroperoxide, is commercially practiced technology. This process is performed in the presence of a solubilized molybdenum catalyst, see U.S. Pat. No. 3,351,635, or a heterogeneous titania on silica catalyst, see U.S. Pat. No. 4,367,342. Another commercially practiced technology is the direct epoxidation of ethylene to ethylene oxide by reaction with oxygen over a silver catalyst. Besides oxygen and alkyl hydroperoxides, another oxidizing agent useful for the preparation of epoxides is hydrogen peroxide. U.S. Pat. No. 4,833,260, for example, discloses the epoxidation of olefins with hydrogen peroxide in the presence of a titanium silicate catalyst. U.S. Pat. No. 6,008,388 describes a direct olefin epoxidation process in which the selectivity for the reaction of olefin, oxygen, and hydrogen in the presence of a noble metal-modified titanium zeolite is enhanced by the addition of a nitrogen compound such as ammonium hydroxide to the reaction mixture. The teachings and disclosures of each of the foregoing patents are incorporated herein by reference in their entirety. While the primary product formed via such epoxidation reactions is the alkylene oxide, the process can produce various side products such as water, acetic acid, formic acid, acetaldehyde, and the like. These side products can be detrimental to processes of cyclic carbonate formation if they remain present in the alkylene oxide feed stream, and further detrimental to the transesterification reaction. In particular, water formed because of the epoxidation reaction is readily capable of reacting with the cyclic carbonate product and/or alkylene oxide reactant.

As such, the purity of each of the reactant feed streams for both the cyclic carbonate formation reaction and the transesterification reaction can be important to the success of such processes. Unfortunately, meeting such purity goals can cause various processing burdens and/or economic infeasibility rendering such processes difficult to implement on commercial scales.

Thus, there is a need to develop processes for forming dialkyl carbonates, where the processes exhibit tolerability to potential impurities present in the reactant feed streams. Further such processes should exhibit reaction rates, product yields, and economic and/or environmental feasibility for commercial implementation thereof into new or existing process plant designs.

### SUMMARY

The present disclosure relates to the production of dialkyl carbonates and glycolic compounds.

In some embodiments, a process includes: (a) epoxidizing an olefin in the presence of a first catalyst to produce a first product stream comprising an alkylene oxide; (b) reacting at least a portion of the first product stream with carbon dioxide in the presence of a second catalyst to produce a second product stream comprising a cyclic carbonate, wherein the second product stream has a water content less than about 0.5 wt % based on the second product stream; and (c) reacting at least a portion of the second product stream with an alcohol in the presence of a third catalyst to produce a third product stream comprising a dialkyl carbonate.

In some embodiments, a process includes providing a first composition comprising an alkylene oxide and at least one of: water and glycol, wherein the first composition has a water content of less than about 5 wt % based on the first composition and a glycol content less than about 5 wt % based on the first composition; and introducing carbon dioxide to the first composition in the presence of a catalyst to produce a second composition comprising a cyclic carbonate and a water content less than about 0.5 wt % based on the second composition, wherein the second composition comprises the catalyst in an amount of about 0.1 wt % to about 5 wt % based on the second composition, wherein producing the second composition comprises converting about 80% or greater of the alkylene oxide to a combination of glycol and the cyclic carbonate.

In some embodiments, a process includes providing a first composition comprising an alkylene oxide, and at least one of: water and glycol, wherein the first composition has a water content of less than about 5 wt % based on the first composition and a glycol content less than about 5 wt % based on the first composition, carbon dioxide, and a first catalyst to a first reactor to produce a second composition comprising a cyclic carbonate, wherein an initial molar ratio of carbon dioxide to alkylene oxide in the first composition is from about 25:1 to about 1:1; and introducing the second composition, an alcohol, and a second catalyst to a second reactor to produce a third composition comprising a dialkyl carbonate.

### BRIEF DESCRIPTION OF DRAWINGS

FIG. 1 is a process flow diagram illustrating a process of simultaneous formation of dialkyl carbonates and mono-glycolic compounds, according to some embodiments.

FIG. 2 is a process flow diagram illustrating a process of simultaneous formation of dialkyl carbonates and mono-glycolic compounds, according to some embodiments.

FIG. 3 is a process flow diagram illustrating a process of simultaneous formation of dialkyl carbonates and mono-glycolic compounds, according to some embodiments.

FIG. 4 is a graph illustrating reaction conversion of reacting an alkylene oxide and carbon dioxide using various catalyst concentrations to form a cyclic carbonate as a function of water content, according to some embodiments.

FIG. 5 is a graph illustrating reaction conversion of reacting an alkylene oxide and carbon dioxide using various catalyst concentrations to form a cyclic carbonate as a function of water content, according to some embodiments.

FIG. 6 is a graph illustrating selectivity as a function of water content, according to some embodiments.

FIG. 7 is a graph illustrating reaction selectivity of reacting an alkylene oxide and carbon dioxide using various catalyst concentrations to form a cyclic carbonate as a function of water content, according to some embodiments.

FIG. 8 is a graph illustrating reaction conversion of reacting an alkylene oxide and carbon dioxide using various reactant ratios to form a cyclic carbonate as a function of water content, according to some embodiments.

FIG. 9 is a graph illustrating reaction conversion of reacting an alkylene oxide and carbon dioxide using various reactant ratios to form a cyclic carbonate as a function of water content, according to some embodiments.

FIG. 10 is a graph illustrating reaction conversion of reacting an alkylene oxide and carbon dioxide using various reaction times to form a cyclic carbonate as a function of mono-glycol content, according to some embodiments.

FIG. 11 is a graph illustrating the effects known impurities have on the reaction conversion of reacting an alkylene oxide and carbon dioxide to form a cyclic carbonate, according to some embodiments.

FIG. 12 is a graph illustrating the effects of using a crude alkylene oxide stream on the reaction conversion of reacting an alkylene oxide and carbon dioxide to form a cyclic carbonate, according to some embodiments.

FIG. 13 is a graph illustrating the effects of using a crude alkylene oxide stream on the reaction selectivity of reacting an alkylene oxide and carbon dioxide to form a cyclic carbonate, according to some embodiments.

FIG. 14 is a graph illustrating the effects water content and catalyst concentration have on the formation of a dialkyl carbonate, according to some embodiments.

FIG. 15 is a graph illustrating the effects water content and catalyst concentration have on the formation of a dialkyl carbonate, according to some embodiments.

FIG. 16 is a graph illustrating the effects water content and catalyst composition have on the formation of dialkyl carbonates, according to some embodiments.

FIG. 17 is a graph illustrating the effects known impurities have on the formation of a dialkyl carbonate using different reactant ratios, according to some embodiments.

FIG. 18 is a graph illustrating the effects known impurities have on the formation of dialkyl carbonate, according to some embodiments.

### DETAILED DESCRIPTION

Processes disclosed herein provide methods of simultaneous formation of dialkyl carbonates and mono-glycolic compounds via the integration of dialkyl carbonate formation processes into commercially implemented pathways of glycolic compound production. Processes disclosed herein implement crude product streams of epoxidation reactions in the formation of cyclic carbonates, where the crude product streams from the formation of cyclic carbonates are implemented as a feed stream for the transesterification reaction. Separation processes described herein allow for an operator to separate dialkyl carbonates from mono-glycolic compounds formed via the transesterification reaction. Reactions and processes disclosed herein are shown to be tolerable to impurities and by-products which may develop during operation. Such reactions and reactant feed streams exemplify economic feasibility of integrating such dialkyl formation processes into commercially implemented pathways of glycol production.

### Processes and Conditions

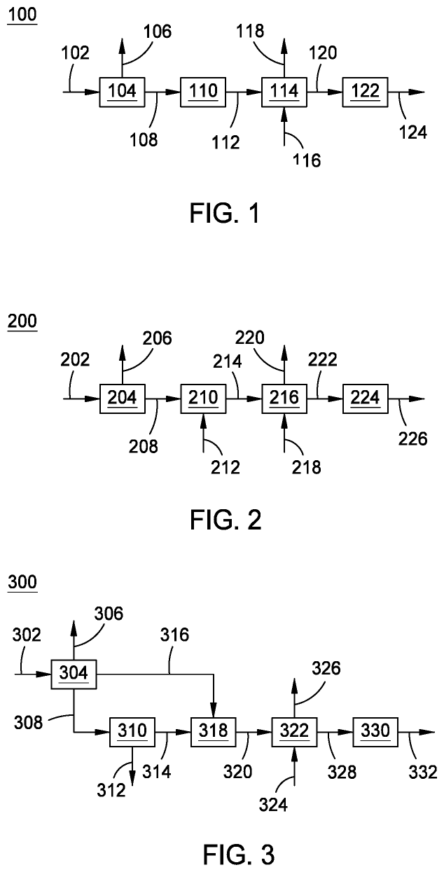

Generally, processes disclosed herein can be illustrated by FIG. 1, wherein one or more olefin compounds are introduced (via line 102) to an epoxidation reactor system 104 having an oxidation catalyst incorporated therein. Such reactions may produce one or more alkylene oxides, which may be at least partially separated from the crude product stream via any suitably implemented separation method, such as a series of distillation processes (line 106 shown removing distillation product which allows production of crude product stream having alkylene oxide of line 108). The remaining crude product stream may be introduced to a reactor system 110 via line 108, where one or more cyclic carbonates may be formed by reacting the one or more alkylene oxides of the crude product stream with CO₂. Upon formation of the one or more cyclic carbonates, the crude product stream of the reactor system 110 may be introduced to a transesterification reactor system 114 via line 112. In some embodiments, in the transesterification reactor system 114, one or more aliphatic monohydric alcohols (via line 116) is introduced to the crude product stream of line 112 in the presence of a transesterification catalyst to produce, at least, one or more dialkyl carbonates and one or more glycols. In at least one embodiment, the one or more dialkyl carbonates may be at least partially removed from the crude product stream of the transesterification reactor system 114 via line 118. Such removal processes may include any one or more suitable processes, such as a series of distillation processes. The remaining crude product stream of the transesterification may be introduced to a separation apparatus 122 via line 120. In some embodiments, separation apparatus 122 may be equipped with one or more of a distillation separating apparatus, an extractive distillation separating apparatus, a liquid-liquid extraction separating apparatus, a crystallization separating apparatus, an absorption separating apparatus, and/or a membrane separating apparatus so as to obtain a mono-glycolic and/or dialkyl carbonate products therefrom (line 124).

In some embodiments, in epoxidation reactor 104, an olefin is reacted with hydrogen and oxygen in the presence of an epoxidation catalyst comprising a transition metal zeolite and/or a noble metal to produce a product stream comprising one or more alkylene oxides and/or one or more alkanes.

In some embodiments, the olefinic compound being epoxidized via the epoxidation reaction is any organic olefinic compound having at least one olefinic double bond, and/or is of the general formula R₁R₂C═CR₃R₄, where each of R₁, R₂, R₃, and R₄ is independently selected from hydrogen, alky, alkenyl, aryl, alkaryl, cycloalkyl, or alkylcycloalkyl, and hydrocarbyl groups. In some embodiments, R₁, R₂, R₃, and R₄ each include no more than 30 carbon atoms, such as no more than 14 carbon atoms. In one or more embodiments, any of R₁, R₂, R₃, and R₄ may be joined together to form a substituted or non-substituted ring structure, such as a cycloalkyl, a cycloalkenyl, a alkylcycloalkyl, a cyclic hydrocarbyl, or an aromatic ring structure. Such substituted or non-substituted ring structure(s) may include 5 to 14 carbon atoms, such as 5 to 12 carbon atoms. In some embodiments, the olefinic compound may be a straight-chain, a branched-chain, or a cyclic olefinic compound. In at least one embodiment, the olefinic compound is a cyclic olefinic compound selected from a monocyclic compound, a bicyclic compound, or a polycyclic compound. In one or more embodiments, the olefinic compound may include one or more olefinic bonds. In such instances where the olefinic compound has more than one olefinic bond, the olefinic compound may be conjugated or non-conjugated. In one or more embodiments, the olefinic compound may include one or more functional groups, such as a halide (e.g., Cl, F, Br, I), a carboxyl, a hydroxyl, an ether, a carbonyl, a cyano, a nitro, or combinations thereof.

In some embodiments, the olefinic compound being epoxidized via the epoxidation reaction is an aliphatic or alicyclic hydrocarbon containing 2 to 20 carbon atoms, such as 2 to 8 carbon atoms, such as 2 to 4 carbon atoms, such as 2 to 3 carbon atoms. In some embodiments, the olefinic compound being epoxidized contains a single double bond.

In some embodiments, the olefinic compound being epoxidized via the epoxidation reaction is selected from ethylene, propylene, butene and isomers thereof, 1-octene, butadiene, allyl chloride, allyl alcohol, styrene, and combinations thereof. In at least one embodiment, the the olefinic compound being epoxidized is ethylene, propylene, or a combination thereof.

Zeolites generally contain one or more of Si, Ge, Al, B, P, or the like, in addition to oxygen. A transition metal zeolite (e.g., titanium zeolite, vanadium zeolite) is a crystalline material having a porous molecular sieve structure and containing a transition metal. A transition metal is a Group 3-12 element, such as Ti, V, Mn, Fe, Co, Cr, Zr, Nb, Mo, and W. The transition metal zeolite employed depends upon a number of factors, including the size and shape of the olefin to be epoxidized. For example, it may be advantageous to use titanium silicalite-1 (TS-1, a titanium silicalite having an MFI topology analogous to that of the ZSM-5 aluminosilicate) for the epoxidation of propylene and/or other olefin compounds. For a bulky olefin such as cyclohexene, larger pore zeolites may be preferred.

In one or more embodiments, suitable titanium zeolites of the present disclosure may include titanium silicates (e.g., titanosilicates). It may be preferable, in some instances, that the suitable titanium zeolites contain no element other than titanium, silicon, and oxygen in the lattice framework (see R. Szostak, “Non-aluminosilicate Molecular Sieves,” in Molecular Sieves: Principles of Synthesis and Identification, (1989), Van Nostrand Reinhold, pp. 205-282). Small amounts of impurities (e.g., boron, iron, aluminum, phosphorous, copper, mixtures thereof, and the like) may be present within the lattice. In some embodiments, the amount of impurities is about 0.5 wt % or less, such as about 0.1 wt % or less. In some embodiments, titanium silicates of the present disclosure may have a composition corresponding to the formula xTiO₂₍ₗ₋ₓ₎SiO₂, where x includes a numerical range of about 0.0001 and about 0.5, such as from about 0.01 to about 0.125. In some embodiments, the molar ratio of Si:Ti in the lattice framework of the zeolite is from about 9.5:1 to about 99:1, such as from about 9.5:1 to about 60:1. In one or more embodiments, it may also be desirable to use of relatively titanium-rich zeolites. In one or more embodiments, the titanium zeolites of the present disclosure may include the class of molecular sieves commonly known as titanium silicalites (as described in Catal. Rev. -Sci. Eng. 39(3) (1997) 209, which is incorporated herein by reference), such as, but not limited to, TS-1, TS-2 (having an MEL topology analogous to that of the ZSM-11 aluminosilicate), and TS-3 (as described in Belgian Pat. No. 1,001,038). In at least one embodiment, the titanium zeolites may include framework structures suitable for use, such as framework structures that are isomorphous to zeolite beta, mordenite, and/or ZSM-12.

In some embodiments, the epoxidation catalyst may additionally, or alternatively, include one or more noble metals, such as gold, silver, platinum, palladium, iridium, ruthenium, osmium, rhenium, rhodium, combinations thereof, and the like. In at least one embodiment, the one or more noble metals are selected from Pd, Pt, Au, Re, Ag, and combinations thereof. In some embodiments, the amount of noble metal present in the catalyst will range from about 1 wt % to about 70 wt %, such as about 30 wt % to about 60 wt %. In some embodiments, the source of the noble metal may include any one or more noble metal compounds and/or complexes, such as nitrates, sulfates, halides, carboxylates, amine and/or phosphine complexes of noble metals, and combinations thereof.

In some embodiments, the epoxidation catalyst includes one or more noble metals and one or more transition metal zeolites. In one or more embodiments, the noble metal(s) and transition metal zeolite(s) may be on a single particle, or on separate ones. For example, the noble metal(s) may be supported on the transition metal zeolite(s). Alternatively, the epoxidation catalyst comprises a mixture of the transition metal zeolite(s) and the noble metal(s), wherein the noble metal(s) may be essentially elemental (e.g., colloidal Pd) or may be supported on a carrier. In one or more embodiments, suitable carriers for the supported noble metal(s) include carbon, titania, zirconia, niobia, silica, alumina, silica-alumina, titania-silica, zirconia-silica, niobia-silica, ion-exchange resins, mixtures thereof, and the like.

In some embodiments, wherein the epoxidation catalyst includes one or more noble metals and one or more transition metal zeolites, where the weight ratio between the two components (zeolite:noble metal) is about 5 g zeolite/1 g noble metal to about 1000 g zeolite/1 g noble metal, such as about 10 g zeolite/1 g noble metal to about 500 g zeolite/1 g noble metal, such as about 20 g zeolite/1 g noble metal to about 100 g zeolite/1 g noble metal.

In some embodiments, an alkyl hydroperoxide may be an oxidizing agent used in the epoxidation reaction. In some embodiments, the alkyl hydroperoxide may be ethylbenzene hydroperoxide or tert-butyl hydroperoxide. In some embodiments, hydrogen peroxide may be an oxidizing agent used in the epoxidation reaction. In some embodiments, oxygen and hydrogen are components used for the epoxidation reaction. In one or more embodiments, the oxygen and/or hydrogen source(s) may be provided via any one or more suitable sources known to one of ordinary skill in the art. In at least one embodiment, the oxygen and hydrogen sources are molecular oxygen and hydrogen. In one or more embodiments, the molar ratio of hydrogen to oxygen introduced to the epoxidation reaction is about 1:10 to about 5:1, such as about 1:5 to about 2:1. In some embodiments, the molar ratio of oxygen to olefin introduced to the epoxidation reaction is about 2:1 to about 1:20, such as about 1:1 to about 1:10, such as about 1:1 to about 1:3.

In some embodiments, the epoxidation reaction can further include the introduction of an inert gas into the reactor in addition to the olefin, oxygen, hydrogen, and epoxidation catalyst. Suitable inert gases can include nitrogen, helium, argon, saturated hydrocarbons, and carbon dioxide. In some embodiments, the inert gas introduced to the epoxidation reaction is nitrogen and/or one or more saturated hydrocarbons having 1 to 8 carbon atoms, such as 1 to 6 carbon atoms, such as 1 to 4 carbon atoms. In at least one embodiment, the saturated hydrocarbons may include methane, ethane, propane, n-butane, and combinations thereof. In some embodiments, the molar ratio of olefin to inert gas introduced to the epoxidation reaction is about 100:1 to about 1:10, such as about 20:1 to about 1:10.

In some embodiments, the epoxidation reaction incorporates the use of a solvent or a solvent mixture that is a liquid under epoxidation reaction conditions. In one or more embodiments, the solvent or solvent mixture of the epoxidation reaction can include one or more oxygen-containing hydrocarbons, one or more aromatic solvents, one or more aliphatic solvents, one or more chlorinated aromatic solvents, one or more chlorinated aliphatic solvents, one or more nitrile based solvents, carbon dioxide, or water. In at least one embodiments, the solvent or solvent mixture of the epoxidation reaction can include one or more of toluene, hexane, chlorobenzene, methylene chloride, acetonitrile, methanol, ethanol, isopropanol, t-butanol, ethers, esters, ketones, carbon dioxide, water, or any monophasic combination thereof.

In some embodiments, it may be advantageous to use a buffer solution or buffer agent as a component of the epoxidation reaction. Without being bound by theory, the buffer solution/buffer agent is employed with the intention of improving aspects of the epoxidation reaction, such as inhibiting glycol and glycol ether formation, increasing reaction rate, increasing reaction selectivity, and the like. In one or more embodiments, the buffer agent may be added to a solvent to form a buffer solution, which can then be introduced to the epoxidation reaction. In one or more embodiments, the buffer agent may be introduced directly to the epoxidation reaction. In some embodiments, buffer agents may include salts of oxyacids, the nature and proportions of which in the mixture are such that the pH of their solutions ranges from about 3 to about 12, such as about 4 to about 10, such as about 5 to about 9. In one or more embodiments, salts of oxyacids include an anion and a cation, wherein the anion may include a phosphate, a carbonate, a bicarbonate, a sulfate, a carboxylate, a borate, a hydroxide, a silicate, a aluminosilicate, or the like. In one or more embodiments, the cation of the oxyacid may be ammonium, alkylammonium, alkylphosphonium, alkali metal, and alkaline earth metal ions, or the like, such as NH₄, NBu₄, NMe₄, Li, Na, K, Cs, Mg, and Ca cations. In some embodiments, the buffer solution may include a combination of salts. In some embodiments, the concentration of the buffer agent in the buffer solution is about 0.0001 M to 1 M, such as about 0.0005 M to 0.3 M. In at least one embodiment, the buffer agent may include one or more of alkali metal phosphates, ammonium phosphate, and/or ammonium hydroxide.

In at least one embodiment, it may be beneficial to introduce a small amount of HBr to the epoxidation reaction, such as about 5 ppm to about 20 ppm based on the total amount of components present in the epoxidation reaction, such as about 8 ppm to about 17 ppm, such as about 10 ppm to about 15 ppm.

In one or more embodiments, epoxidation catalyst may be in the form of a suspension or fixed-bed, such that the epoxidation reaction may be performed in a continuous flow, semi-batch, and/or batch mode. In at least one embodiment, the epoxidation reaction is carried at a suitable temperature and pressure to form an alkylene oxide from an olefin compound. In some embodiments, the epoxidation reaction is carried out at a pressure of about 1 bar to about 200 bars, such as about 2 bars to about 100 bars, such as about 5 bars to about 50 bars. In some embodiments, the epoxidation reaction is carried out at a temperature of about 0 °C to about 200 °C, such as about 50 °C to about 170 °C. In at least one embodiment, at least a portion of the epoxidation reaction mixture is a liquid under the prescribed epoxidation reaction conditions.

In some embodiments, the epoxidation reaction may be carried out using appropriate temperature, pressure, and reactant ratios necessary to achieve the desired reaction. In one or more embodiments, the epoxidation reaction is carried out at a temperature of about 50 °C to about 250 °C, such as about 100 °C to about 225 °C, such as about 100 °C to about 200 °C. In some embodiments, the epoxidation reaction is carried out at a pressure of about 1 bars to about 300 bars, such as about 25 bars to about 250 bars, such as about 50 bars to about 200 bars. In some embodiments, the molar ratio of hydrogen to oxygen is about 1:100 to about 5:1, such as about 1:50 to about 2:1, such as about 1:5 to about 2:1. In some embodiments, the epoxidation catalyst is added to the epoxidation reaction at a feed rate sufficient to provide a transition metal/olefin per hour molar feed ratio of about 0.0001 to about 0.1, such as about 0.001 to about 0.1, such as about 0.01 to about 0.05. In some embodiments, the molar ratio of olefin to inert gas introduced to the epoxidation reaction is about 100:1 to about 1:10, such as about 20:1 to about 1:10, such as about 10:1 to about 1:10. In some embodiments, the weight ratio between the one or more noble metals and one or more transition metal zeolites is about 0.01 g zeolite/1 g noble metal to about 100 g zeolite/1 g noble metal, such as about 0.1 g zeolite/1 g noble metal to about 100 g zeolite/1 g noble metal, such as about 1 g zeolite/1 g noble metal to about 1 g zeolite/1 g noble metal. In some embodiments, the epoxidation reaction is performed as a continuous reaction process or a batch reaction process. In some embodiments, the reaction time of the epoxidation reaction is about 1 minute to about 24 hours, such as about 1 minute to about 6 hours, such as about 1 minute to about 1 hour.

In some embodiments, the epoxidation reaction of reactor system 104 produces a crude product stream that can include, at least, one or more of an alkylene oxide, a transition metal zeolite, a noble metal compound, water, CO₂, glycols, small aldehydes (formaldehyde, acetaldehyde), and small organic acids (e.g., formic acid, acetic acid). In some embodiments, the alkylene oxide may be, at least partially, separated from the crude product stream of the epoxidation reaction via any one or more methods readily apparent to one of ordinary skill in the art. In some embodiments, the crude product stream of the epoxidation reaction may be passed through a filter to remove solid particulates therefrom. In some embodiments, light materials present in the crude product stream of the epoxidation reaction (e.g., gaseous alkanes, alkenes, oxygen, hydrogen, and other inert gases) may be separated therefrom via any suitable one or more methods, such as an evaporation process and/or a distillation process. In one or more embodiments, the remaining components of the crude epoxidation reaction product stream may undergo further distillation to, at least partially, separate the alkylene oxide therefrom. In at least one embodiment, the remaining crude product stream of the epoxidation reaction may be considered as a crude product stream (ethylene oxide or propylene oxide), which may be transferred and/or introduced to a reaction composition to produce cyclic carbonates therefrom. In some embodiments, the crude product stream of the epoxidation reaction includes a water content of about 50 wt % or less of the reaction composition, such as about 25 wt %, 15 wt %, 10 wt %, 5 wt %, 3.5 wt %, 2 wt % or less. In some embodiments, crude product stream of the epoxidation reaction includes a water content of about 0.01 wt % to about 5 wt % of the reaction composition, such as about 0.05 wt % to about 2.5 wt %, such as about 0.25 wt % to about 2 wt %, such as about 0.25 wt % to about 1 wt %, such as about 0.25 wt % to about 0.5 wt %.

In some embodiments, the reactor system 110 is designed/composed for the formation of cyclic carbonates carried out via reacting alkylene oxide with carbon dioxide. Reactions and processes resulting in formation of cyclic carbonates from epoxides have previously been investigated and established in prior art (e.g., U.S. Pat. Nos. 2,773,070, 2,873,282, 4,786,741, 5,179,214, and 5,283,356). However, processes disclosed herein are differentiated from such reports through integration/implementation of crude product streams of the epoxidation reaction as the source of the alkylene oxide from which the cyclic carbonate is formed. Without being bound by theory, such integration/implementation of crude product streams (ethylene oxide or propylene oxide) may allow for simplified process designs, enhanced economic impact, and/or increased control and simplification of subsequent reaction and product separation processes. In some embodiments, the crude product streams of the epoxidation reaction may include one or more impurities, such as acetic acid, acetaldehyde, formic acid, formaldehyde and combinations thereof. In some embodiments, the crude product streams may include impurities in an amount of about 1 ppm to about 10,000 ppm of the overall product stream, such as about 25 ppm to about 7,500 ppm, such as about 50 ppm to about 5,000 ppm.

In some embodiments, the formation of cyclic carbonates may be conducted in a similar manner to U.S. Pat. No. 5,283,356, where alkylene oxide present within the crude product streams of the epoxidation reaction is reacted with carbon dioxide, in the presence of a catalyst, at a temperature of about 100 °C to about 225 °C, such as about 125 °C to about 200 °C. In one or more embodiments, the reaction may be carried out at atmospheric pressure or a pressure of about 300 psig or greater, such as about 300 psig to about 3000 psig, such as about 300 psig to about 800 psig. In one or more embodiments, the reaction may be conducted either batch-wise or continuously.

In a continuous reaction process, the crude product streams of the epoxidation reaction 104 (and alkylene oxide contained therein) and carbon dioxide are introduced to a continuous reactor containing the catalyst to form the reaction composition of reactor system 110, from which a portion of the reaction composition may be continuously recirculated through the reactor. Another portion of the reaction composition is continuously withdrawn and treated to remove unreacted alkylene oxide from product cyclic alkylene carbonate. Alternatively, the continuous reaction can be carried out on a once through basis with suitable heat removal.

Alternatively, batches of reaction composition of reactor system 110 may be introduced into an autoclave or kettle reactor. The desired pressure may be built up by introducing carbon dioxide. Typically, the reaction mixture is heated to reaction temperature, agitated, and held under a super atmospheric pressure of carbon dioxide. After the bulk of the alkylene oxide has reacted, the reaction mixture can be treated to remove unreacted alkylene oxide.

Although the alkylene oxide is provided via crude product streams of the epoxidation reaction of reactor system 104, the alkylene oxide and carbon dioxide should be mixed in proportion to provide an excess of carbon dioxide over and above the stoichiometric amount for reaction. In some embodiments, the excess may be about 1.1 moles of carbon dioxide per mole of alkylene oxide to about 10 moles of carbon dioxide per mole of alkylene oxide. In some embodiments, the excess may be about 2 moles of carbon dioxide per mole of alkylene oxide. In some embodiments, the excess may be about 3 moles of carbon dioxide per mole of alkylene oxide. In some embodiments, the excess may be about 4 moles of carbon dioxide per mole of alkylene oxide. An excess of alkylene oxide should be avoided, because it results in undesired by-products, such as rapid alkylene oxide formation.

In some embodiments, the catalyst used in the reaction composition of reactor system 110 to form cyclic carbonates may be a composition of one or more homogenous catalyst compounds and/or one or more heterogeneous catalyst compounds. In some embodiments, the catalyst used in the cyclic carbonate formation may include combinations of zinc halides (e.g., zinc iodide and zinc bromide) with quaternary ammonium or phosphonium halides (e.g., n-butyl ammonium halides), ionic liquids (e.g., imidazolium salts), pyridine derivatives, indium halides, lead halides, polyoxometalates, alkali metal halides (e.g., potassium iodide and potassium bromide), and halogenated organic phosphonium or ammonium salts (e.g., tributylmethylphosphonium iodide, tetrabutylphosphonium iodide, triphenylmethylphosphonium iodide, triphenylpropylphosphonium bromide, triphenylbenzylphosphonium chloride, tetraethylammonium bromide, tetramethylammonium bromide, benzyltriethylammonium bromide, tetrabutylammonium bromide, and tributylmethylammonium iodide). In some embodiments, the catalyst used in the cyclic carbonate formation may be based upon solid supports such as ion exchange resins, silica, polysiloxane, polyvinylpyridine and/or polystyrene. In one or more embodiments, a solid support, such as an ion exchange resin, may be functionalized with a quaternary ammonium or phosphonium halide and used in combination with a metal salt co-catalyst, such as a zinc halide. In at least one embodiment, quaternary ammonium and/or quaternary phosphonium halides may be immobilized on silica or bound to insoluble polystyrene beads. In at least one embodiment, metal salts (e.g., zinc halides) may be supported on solid supports, such as polyvinylpyridine, polyvinylpyrrolidine, and/or chitosan. In at least one embodiment, the catalyst is tetraethylammonium bromide (TEAB), potassium iodide, or a combination thereof.

In some embodiments, the reaction composition of reactor system 110 to form the cyclic carbonate includes a molar ratio of CO₂ to alkylene oxide of about 25:1 to about 1:1, such as about 15:1 to about 1:1, such as about 5:1 to about 1:1, such as about 4:1 to about 1:1. In some embodiments, the reaction composition of reactor system 110 to form the cyclic carbonate includes a catalyst in an amount of about 0.01 wt % to about 5 wt % of the total reaction composition, such as about 0.05 wt % to about 2.5 wt %, such as about 0.1 wt % to about 1 wt %, such as about 0.25 wt % to about 0.5 wt %. In some embodiments, the reaction to form the cyclic carbonate is conducted at a temperature (e.g., reactor temperature) of about 100 °C to about 250 °C, such as about 130 °C to about 225 °C, such as about 150 °C to about 200 °C. In some embodiments, the reaction to form the cyclic carbonate is conducted using a pressure (e.g., reactor pressure) from about 300 psig to about 3,000 psig, such as about 400 psig to about 2,000 psig, such as about 500 psig to about 800 psig. In some embodiments, the reaction composition to form the cyclic carbonate includes a water content provided by the epoxidation reaction. In at least one embodiment, a water content is provided to the reaction composition via an external water source. In some embodiments, the reaction composition to form the cyclic carbonate includes a water content of about 50 wt % or less of the reaction composition, such as about 25 wt %, 15 wt %, 10 wt %, 5 wt %, 3.5 wt %, 2 wt % or less. In some embodiments, the reaction composition to form the cyclic carbonate includes a water content of about 0.01 wt % to about 5 wt % of the reaction composition, such as about 0.05 wt % to about 2.5 wt %, such as about 0.25 wt % to about 2 wt %, such as about 0.25 wt % to about 1 wt %, such as about 0.25 wt % to about 0.5 wt %. In some embodiments, the reaction to form the cyclic carbonate is performed at a reaction time of about 10 minutes to about 600 minutes, such as about 20 minutes to about 200 minutes, such as about 30 minutes to about 150 minutes.

In some embodiments, the cyclic carbonate formation reaction reaches an alkylene oxide reactant conversion of about 50% or greater, such as about 75% or greater, such as about 90% or greater, such as about 95% or greater, such as about 97.5% or greater, such as about 99% or greater, such as about 99.9% or greater. In some embodiments, the cyclic carbonate formation reaction includes a reaction selectivity of about 80% or greater, such as about 85% or greater, such as about 90% or greater, such as about 95% or greater, such as about 97.5% or greater, such as about 99% or greater, such as about 99.9% or greater.

In some embodiments, upon completion of the desired reaction between the alkylene oxide and carbon dioxide to form alkylene carbonate, the reaction composition of reactor system 110 may be treated to remove residual unreacted alkylene oxide. As such, the system pressure is reduced and carbon dioxide and alkylene oxide are vented from the system. In some embodiments, even after such venting, the reaction mixture may contain high levels of alkylene oxide, such as about 0.1 wt % or greater. Simple flashing and removal of flashed alkylene oxide and carbon dioxide is generally ineffective in reducing the level of alkylene oxide to an acceptable amount.

In at least one embodiment, the stripping procedure of U.S. Pat. No. 5,631,386 may be implemented to achieve acceptable levels of residual alkylene oxide. As such, the temperature of the reaction mixture is reduced and stripped of residual alkylene oxide by passage of an inert gas there through until the residual alkylene oxide level is about 1 wt % or less, such as about 0.5 wt % or less, such as about 0.1 wt % or less, such as about 0.01 wt % or less. Generally, inert gas vapor is passed (e.g., bubbled) through the reaction composition at a rate of about 0.01 to about 1 parts by weight of inert gas vapor per parts by weight liquid per hour, such as about 0.02 to about 0.2 parts by weight of inert gas vapor per parts by weight liquid per hour. Generally, such stripping procedures are conducted for about 5 minutes to about 200 minutes, such as about 10 minutes to about 100 minutes.

In some embodiments, the crude product stream of the reaction composition of reactor system 110 is introduced (via line 112) to a transesterification reactor system 114 to produce one or more dialkyl carbonates and/or one or more glycols.

In some embodiments, the transesterification reaction of reactor system 114 may be carried out via reacting the cyclic carbonate content present in the crude product stream of the reaction composition of line 112 with one or more aliphatic monohydric alcohols in the presence of a catalyst. In some embodiments, the one or more aliphatic monohydric alcohols may be introduced to the transesterification reaction of reactor system 114 via line 116.

In some embodiments, the one or more aliphatic monohydric alcohols is selected from methanol, ethanol, n-propanol, isopropanol, alkyl alcohol, butanol and isomers thereof, 3-butene-1-ol, amyl alcohol and isomers thereof, hexyl alcohol and isomers thereof, heptyl alcohol and isomers thereof, octyl alcohol and isomers thereof, nonyl alcohol and isomers thereof, decyl alcohol and isomers thereof, undecyl alcohol and isomers thereof, dodecyl alcohol and isomers thereof, cyclopentanol, cyclohexanol, cycloheptanol, cyclooctanol, methylcyclopentanol and isomers thereof, ethylcyclopentanol and isomers thereof, methylcyclohexanol and isomers thereof, ethylcyclohexanol and isomers thereof, dimethylcyclohexanol and isomers thereof, diethylcyclohexanol and isomers thereof, phenylcyclohexanol and isomers thereof, benzyl alcohol, phenethyl alcohol and isomers thereof, phenylpropanol and isomers thereof, any combination(s) thereof, and the like. In some embodiments, the aliphatic monohydric alcohol may be substituted with at least one substituent, such as a halogen atom, a lower alkoxy group, a cyano group, an alkoxycarbonyl group, an aryloxycarbonyl group, an acyloxy group, a nitro group or the like. In one or more embodiments, the aliphatic monohydric alcohol is an alcohol having 1 to 6 carbon atoms, such as 1 to 4 carbon atoms, such as methanol, ethanol, propanol, butanol or isomers thereof.

Without being bound by theory, the transesterification reaction between the cyclic carbonate crude product stream of the crude product of reactor system 110 and the aliphatic monohydric alcohol involves two equilibrium steps which include the formation of a hydroxyalkyl carbonate as the reaction intermediate. The two equilibrium step reactions may be represented by the following Scheme 1:

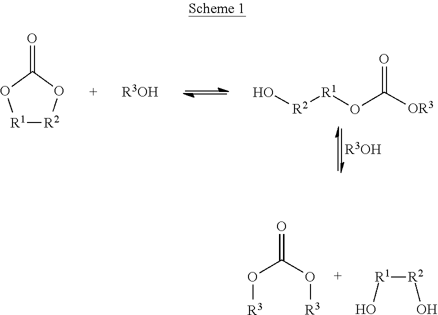

where R¹ and R² are each independently an unsubstituted carbon or substituted carbon having a substituent with no more than 30 carbon atoms bonded thereto, such as no more than 14 carbon atoms, such as no more than 8 carbon atoms. In some embodiments, substituents on the one or more substituted carbons may be a straight-chain, a branched-chain, or a cyclic olefinic compound. In one or more embodiments, R¹ and R², may be joined together to form a substituted or non-substituted ring structure, such as a cycloalkyl, a cycloalkenyl or a alkylcycloalkyl, a cyclic hydrocarbyl, or an aromatic ring structure. Such substituted or non-substituted ring structure(s) may include 5 to 14 carbon atoms, such as 5 to 12 carbon atoms. R₃ is an aliphatic C₁-C₁₂ hydrocarbon group, which is unsubstituted or substituted with at least one substituent selected from the group consisting of a C₁-C₁₀ alkyl group and/or a C₆-C₁₀ aryl group.

In some embodiments, the reactants to the transesterification reaction (e.g., the cyclic carbonate and the aliphatic monohydric alcohol) are reacted in the presence of a transesterification catalyst. The transesterification catalyst can typically include any homogeneous and/or heterogeneous catalyst known in the art, which provides adequate reaction kinetics and minimizes side reactions with the impurities contained in the cyclic carbonate.

In some embodiments, the transesterification catalyst may include one or more alkali metals or alkaline earth metals, such as lithium, sodium, potassium, rubidium, cesium, magnesium, calcium, strontium, barium and the like. In some embodiments, the transesterification catalyst may include one or more basic compounds such as hydrides, hydroxides, alkoxides, aryloxides and amides of alkali metals or alkaline earth metals and the like. In some embodiments, the transesterification catalyst may include one or more basic compounds, such as carbonates and hydrogencarbonates of alkali metals or alkaline earth metal, alkali metal or alkaline earth metal salts of organic acids and the like. In some embodiments, the transesterification catalyst may include one or more tertiary amines (e.g., triethylamine, tributylamnine, trihexylamine, benzyldiethylamine, and the like), nitrogen containing heteroaromatic compounds (e.g., N-alkylpyrrole, N-alkylindole, oxazole, N-alkylimidazole, N-alkylpyrazole, oxadiazole, pyridine, alkylpyridine, quinoline, alkylguinoline, isoquinoline, alkylisoquinoline, acridine, alkylacridine, phenanthroline, alkylphenanthroline, pyrimidine, alkylpyrimidine, pyradine, alkylpyradine, triazine, alkyltriazine, and the like), cyclic amidines (e.g., diazabicycloundecene (DBU), diazabicy clononene (DBN) and the like), thallium compounds (e.g., thallium oxide, thallium halides, thallium hydroxide, thallium carbonate, thallium nitrate, thallium sulfate, thallium salts of organic acids, and the like), tin compounds (e.g., tributylmethoxytin, tributylethoxytin, dibutyldimethoxytin, diethyldiethoxytin, dibutyldiethoxytin, dibutylphenoxytin, diphenylmethoxytin, dibutyltin acetate, tributyltin chloride, tin 2-ethylhexanoate, and the like), zinc compounds (e.g., dimethoxy zinc, diethoxy zinc, ethylenedioxy zinc, dibutoxyzinc, and the like), aluminum compounds (e.g., aluminum trimethoxide, aluminum triisopropoxide, aluminum tributoxide, and the like), titanium compounds (e.g., tetramethoxy titanium, tetraethoxy titanium, tetrabutoxy titanium, dichlorodimethoxy titanium, tetraisopropoxytitanium, titanium acetate, titanium acetylacetonate, and the like), phosphorus compounds (e.g., trimethylphosphine, triethylphosphine, tributylphosphine, triphenylphosphine, tributylmethylphosphonium halides, trioctylbutylphosphonium halides, triphenylmethylphosphonium halides, and the like), zirconium compounds (e.g., zirconium halides, zirconocenes, zirconium acetylacetonate, zirconium alkoxides, zirconium acetate, and the like), lead oxides (e.g., PbO, PbO₂, Pb₃O₄, and the like), lead sulfides (e.g., PbS, PbS₂, Pb S, and the like), lead hydroxides (e.g., Pb(OH), PbO(OH), Pb—PbO2(OH)), PbO(OH), and the like), plumbites (e.g., Na₂PbO₂, K₂PbO₂, NaHPbO₂, KHPbO₂, and the like), plumbates (e.g., Na₂PbO₃, Na₂H₂PbO₄, K₂PbO₃, K₂[Pb(OH)₆], K₄PbO₄, Ca₂PbO₄, CaPbO₃, and the like), lead carbonates and basic salts thereof (e.g., PbCO₃, PbCO₃·Pb(OH)₂, and the like), alkoxylead compounds and aryloxylead compounds (e.g., Pb(OCH₃)₂, (CH₃O) Pb(OPh), Pb(OPh)₂, and the like), lead salts of organic acids and carbonates and basic salts thereof (e.g., Pb(OCOCH₃)₂, Pb(OCOCH₃)₄, Pb(OCOCH₃)₂·PbO·3H₂O, and the like), organolead compounds (e.g., Bu₄Pb, Ph₄Pb, Bu₄PbC, Ph₃PbBr, Ph₃Pb (or Ph₆Pb₂), Bu₃PbOH, Ph₂PbO, and the like; wherein Bu is indicative of a butyl group and Ph is indicative of a phenyl group), lead alloys (e.g., Pb—Na, Pb—Ca, Pb—Ba, Pb—Sn, Pb—Sb, and the like), lead minerals (e.g., galena, zinc blends, and the like), hydrates of these lead compounds, ion-exchangers (e.g., anion-exchange resins having tertiary amino groups, amide groups, or at least one ion-exchange group selected from the group consisting of sulfonate, carboxylate and phosphate groups, strongly basic solid anion-exchangers having quaternary ammonium groups as ion-exchange groups, and the like), and solid inorganic compounds (e.g., silica, silica-alumina, silica-magnesia, aluminosilicate, gallium silicate, various zeolites, various metal-exchanged zeolites, ammonium-exchanged zeolites, and the like). In at least one embodiment, the transesterification catalyst is potassium methoxide.

In some embodiments, the transesterification reaction may be carried out with a weight ratio of transesterification catalyst to cyclic carbonate of about 0.0005:1 to about 0.05:1, such as about 0.002:1 to about 0.01:1, such as about 0.005:1 to about 0.01:1. In one or more embodiments, the transesterification catalyst is a homogeneous catalyst and is present in a weight ratio relative to the cyclic carbonate in a range of about 0.0005:1 to about 0.05:1, such as about 0.002:1 to about 0.01:1, such as about 0.005:1 to about 0.01:1. In one or more embodiments, the transesterification catalyst is a heterogenous catalyst and is present in a weight ratio relative to the cyclic carbonate in a range of about 0.0005:1 to about 0.05:1, such as about 0.002:1 to about 0.01:1, such as about 0.005:1 to about 0.01:1.

In some embodiments, the transesterification reaction may be carried out in a continuous mode utilizing a single or multiple-reactor configuration utilizing any one or more reactor designs, such as a stirred-tank reactor, a tubular reactor, a fixed and/or packed-bed reactor, a boiling pot surmounted by a trayed or packed column, and/or a reactive distillation column. In some embodiments, the transesterification reaction may be conducted at a temperature range of about 25 °C to about 250 °C, such as about 35 °C to about 200 °C, such as about 40 °C to about 150 °C. In some embodiments, the transesterification reaction may be conducted at a pressure range of about 0 psig to about 2,000 psig, such as about 0 psig to about 1,000 psig, such as about 0 psig to about 300 psig. In some embodiments, the transesterification reaction may be conducted using a residence time of about 1 minute to about 24 hours, such as about 1 minute to about 6 hours, such as about 1 minute to about 1 hours.

In some embodiments, the transesterification reaction may be carried out using a molar ratio of aliphatic monohydric alcohol to cyclic carbonate of about 1:1 to about 50:1, such as about 1:1 to about 15:1, such as about 1:1 to about 8:1. In some embodiments, the reaction composition of the transesterification reaction includes a water content of about 5 wt % or less of the reaction composition, such as about 2 wt % or less, such as about 0.5 wt % or less. In some embodiments, the transesterification reaction may include a water content of about 0 wt % to about 5 wt % of the reaction composition, such as about 0.5 wt % to about 5 wt %, such as about 2 wt % to about 5 wt %. In at least one embodiment, the water content present in the reaction composition of the transesterification reaction may be provided thereto via residual water produced by one or more of the previous reactions (e.g., the epoxidation reaction and the cyclic carbonate formation reaction). In at least one embodiment, the water content present in the reaction composition of the transesterification reaction may be provided thereto via an external water source. In some embodiments, the reaction composition of the transesterification reaction may include one or more impurities provided thereto one or more of the previous reactions, such as acetic acid, acetaldehyde, formic acid, TEAB, formaldehyde and combinations thereof. In some embodiments, the reaction composition of the transesterification reaction may include impurities in an amount of about 1 ppm to about 10,000 ppm of the reaction composition, such as about 5 ppm to about 7,500 ppm, such as about 20 ppm to about 5,000 ppm.

In some embodiments, the transesterification reaction reaches a cyclic carbonate reactant conversion of about 25% or greater, such as about 75% or greater, such as about 90% or greater, such as about 95% or greater, such as about 97.5% or greater, such as about 99% or greater, such as about 99.9% or greater. In some embodiments, the transesterification reaction produces a product stream including about 1 mol % to about 50 mol % of dialkyl carbonate, such as about 1 mol % to about 25 mol %, such as about 1 mol % to about 15 mol %. In some embodiments, the dialkyl carbonate produced can have a symmetry (such as a C1 or C2 symmetry). Without being bound by theory, the symmetry of the product dialkyl carbonate exhibits a dependency on the composition of the aliphatic monohydric alcohol stream introduced to the transesterification reactor 114. For example, if methanol is used, the product dialkyl carbonate will have a C1 symmetry. In some embodiments, the transesterification reaction produces a product stream including about 1 mol % to about 99.9 mol % of glycol, such as about 75 mol % to about 99.9 mol %, such as about 85 mol % to about 99.9 mol %. In some embodiments, the transesterification reaction produces a product stream including one or more glycolic compounds and one or more dialkyl carbonate compounds, where at least one of the glycolic compounds and at least one of the dialkyl carbonate compounds are formed simultaneously.

The mode of operation, reactor design, temperature, and pressure can be optimized to insure a relatively high conversion of the cyclic carbonate to the desired dialkyl carbonate and glycol. In addition, the process may provide economic potential of the overall integrated process. Such aspects should be taken into account when developing and/or designing a transesterification reaction system 114. Generally, a reactive distillation column will tend to give higher conversions of ethylene carbonate and methanol, while a packed bed reactor offers cheaper capital costs.

In some embodiments, the product stream produced from the transesterification reaction may contain, at least, one or more of a dialkyl carbonate, a glycol, an unreacted cyclic carbonate, and/or an unreacted aliphatic monohydric alcohol. As such, it may be desirable to separate the components of the transesterification reaction.

In some embodiments, the product stream produced from the transesterification reaction may be subjected to one or more evaporation/distillation processes (line 118) to separate condensable components therefrom (e.g., product glycol and unconverted alkylene carbonate). In some embodiments, the product stream produced from the transesterification reaction may be introduced to a separation apparatus 122 so as to obtain a mono-glycolic and/or dialkyl carbonate products therefrom (line 124). In some embodiments, components of the product stream produced from the transesterification reaction may exhibit physical properties (e.g., differences in boiling point) that allow for separation techniques to be implemented on the transesterification product of line 120.

In some embodiments, the product stream produced from the transesterification reaction may be transferred to a separation apparatus 122 to separate the dialkyl carbonate product and unreacted aliphatic monohydric alcohol from product glycol and unconverted alkylene carbonate. In some embodiments, a separating apparatus(s) may include one or more distillation separating apparatus, an extractive distillation separating apparatus, a liquid-liquid extraction separating apparatus, a crystallization separating apparatus, an absorption separating apparatus, and/or a membrane separating apparatus. A combination of a plurality of separating apparatuses may be used, where the apparatuses may include different designs, identical designs, or a combination thereof. In at least one embodiment, a distillation separating apparatus is utilized as a single independent separating apparatus or in series with one or more additional separation apparatuses. In at least one embodiment, substantially all of the unreacted aliphatic monohydric alcohol is removed from the reactor effluent stream.

In some embodiments, the product stream of the transesterification reaction includes, at least, the product glycol, the product dialkyl carbonate, unreacted aliphatic monohydric alcohol, and unconverted alkylene carbonate. In at least one embodiment, the product stream of the transesterification reaction may be transferred to a separate separation apparatus 122 and subjected to a separate separation procedure. In some embodiments, the separation may include performing one or more distillation processes. In at least one embodiment, a first distillation process may be conducted to separate the components of the product stream of the transesterification reaction into a first portion and a second portion. In at least one embodiment, the first portion may include, at least, the product dialkyl carbonate and unreacted aliphatic monohydric alcohol. The second portion may include, at least, the product glycol and unconverted alkylene carbonate. Distillation conditions in the first distillation column may include a pressure ranging from about 0.01 bar to about 10 bar, such as about 0.1 bar to about 1.5 bar. Distillation conditions in the first distillation column may include a temperature ranging from about 50 °C to about 300 °C, such as about 50 °C to about 200 °C.

In some embodiments, the first portion (e.g., containing at least the product dialkyl carbonate and unreacted aliphatic monohydric alcohol) separated from the product stream of the transesterification reaction is further separated via one or more additional distillation processes to recover the product dialkyl carbonate, where the one or more additional distillation processes may be conducted using the same or different distillation columns. In at least one embodiment, the separation of the first portion may be conducted in a second distillation column, such that the dialkyl carbonate is separated from the unreacted aliphatic monohydric alcohol. In at least one embodiment, distillation conditions in the second distillation column may include a pressure ranging from about 1 bar to about 50 bar, such as about 1.5 bar to about 30 bar. In at least one embodiment, distillation conditions in the second distillation column may include a temperature ranging from about 50 °C to about 200 °C, such as about 50 °C to about 150 °C.

In at least one embodiment, the components of the first portion of the product stream of the transesterification reaction (e.g., product dialkyl carbonate and unreacted aliphatic monohydric alcohol) may form an azeotrope. In such instances, it may be beneficial to implement extractive distillation using an extractant to facilitate separation of the components of the first portion of the effluent stream. In at least one embodiment, the extractant can include one or more phenolic compounds and/or ether compounds. In at least one embodiment, the parent cyclic carbonate used transesterification reaction may be used as the extractant. In some embodiments, the product dialkyl carbonate separated from the first portion of the effluent stream may be collected, further purified, stored, and/or recycled for use subsequent reactions and/or consumer goods/services. In at least one embodiment, the product dialkyl carbonate may be further purified via one or more distillation and/or ion-exchange processes, as described in U.S. Pat. No. 5,455,368.

In at least one embodiment, the components of the second portion of the product stream of the transesterification reaction (e.g., product glycol and unconverted alkylene carbonate) is further separated via one or more additional distillation processes to recover the product glycol, where the one or more additional distillation processes may be conducted using the same or different distillation columns. In at least one embodiment, the separation of the second portion may be conducted in a third distillation column, such that the product glycol is separated from the unconverted alkylene carbonate. In at least one embodiment, distillation conditions in the third distillation column may include a pressure ranging from about 0.01 bar to about 2 bar, such as about 0.1 bar to about 1 bar. In at least one embodiment, distillation conditions in the third distillation column may include a temperature ranging from about 100 °C to about 400 °C, such as about 150 °C to about 300 °C.

In some embodiments, the process of simultaneous formation of dialkyl carbonates and mono-glycolic compounds can be illustrated by FIG. 2, wherein one or more olefin compounds (e.g. propylene) are introduced (via line 202), to an epoxidation reaction system 204 having an oxidation catalyst incorporated therein. Such reactions may produce one or more alkylene oxides. In some embodiments, the one or more alkylene oxides may be partially separated from the crude product stream of the epoxidation reactor system 204 via any suitable separation method, such as a series of distillation processes (line 206). The remaining crude product stream of the epoxidation reactor system 204 may be introduced to a reactor system 210 via line 208, where one or more cyclic carbonates may be formed by reacting the one or more alkylene oxides of the crude product stream with CO₂ provided via line 212. In some embodiments, the CO₂ used in the cycloaddition reaction is sourced from recycled CO₂ produced as a byproduct of other reaction processes. Upon formation of the one or more cyclic carbonates, the crude product stream of the reactor system 210 may be introduced to a transesterification reactor system 216 via line 214. In some embodiments, the transesterification reactor system 216 includes introducing one or more aliphatic monohydric alcohols (via line 218) to the crude product stream of the reactor system 210 in the presence of a transesterification catalyst to produce, at least, one or more dialkyl carbonates and one or more glycols. In at least one embodiment, the one or more dialkyl carbonates may be at least partially removed from the crude product stream of the transesterification reaction 216 via line 220. Such removal processes may include any suitable one or more processes, such as a series of distillation processes. The remaining crude product stream of the transesterification reactor system 216 may be introduced to a separation apparatus 224 via line 222. In some embodiments, the separation apparatus may be equipped with one or more of a distillation separating apparatus, an extractive distillation separating apparatus, a liquid-liquid extraction separating apparatus, a crystallization separating apparatus, an absorption separating apparatus, and/or a membrane separating apparatus so as to obtain a mono-glycolic and/or dialkyl carbonate products from the product stream of the transesterification reaction (line 226).

In some embodiments, the processes, parameters, and/or reactants of the epoxidation reactor system 204 may include any such processes, parameters, and/or reactants previously disclosed, such as those disclosed for the epoxidation reaction of reactor system 104. In some embodiments, the processes, parameters, and/or reactants of the reactor system 210 used to form cyclic carbonates from the reaction of one or more alkylene oxides with CO₂ may include any such processes, parameters, and/or reactants previously disclosed, such as those disclosed for the reactor system 110. In some embodiments, the processes, parameters, and/or reactants of the transesterification reactor system 216 may include any such processes, parameters, and/or reactants previously disclosed, such as those disclosed for the transesterification reactor system 114. In some embodiments, the processes, parameters, and/or apparatuses of the separation apparatus 224 used to separate components of the crude product stream of the transesterification reactor system 214 may include any such processes, parameters, and/or apparatuses previously disclosed, such as the disclosed for the separation apparatus 122.

In some embodiments, the process of simultaneous formation of dialkyl carbonates and mono-glycolic compounds can be illustrated by FIG. 3, wherein one or more olefin compounds (e.g., ethylene) are introduced (via line 302) to an epoxidation reactor system 304, having an oxidation catalyst incorporated in the reactor. Such reactions may produce one or more alkylene oxides, water, and CO₂. In some embodiments, the one or more alkylene oxides may be at least partially separated from the crude product stream of the epoxidation reactor system 304 via any suitable separation method, such as a series of distillation processes (line 306). In some embodiments, the CO₂ gas may be vented/removed from the epoxidation reactor system 304, and recycled and/or reintegrated into various other reactions and/or processes (e.g., via line 316). The remaining crude product stream of the epoxidation reactor system 304 may be introduced to a water removal apparatus 310 via line 308, wherein at least a portion of the water content present is removed from the crude product stream of the epoxidation reactor system 304 via line 312. In at least one embodiment, the water removed therefrom is recycled and/or suitably treated for use in one or more processes, reactions, and/or consumer products. Upon reduction of water content to a suitable/desired level, the remaining crude product stream of the epoxidation reactor system 304 may be introduced to a reactor system 318 via line 314, where one or more cyclic carbonates may be formed by a cycloaddition reaction between the one or more alkylene oxides of the crude product stream and CO₂. In some embodiments, the CO₂ used in the cycloaddition reaction is sourced from the recycled CO₂ produced from the epoxidation reactor system 304 via line 316. Upon formation of the one or more cyclic carbonates, the crude product stream of the reactor system 318 may be introduced to a transesterification reactor system 322 via line 320. In some embodiments, the transesterification reaction includes introducing one or more aliphatic monohydric alcohols (via line 324) to the crude product stream of the reaction reactor system 318 in the presence of a transesterification catalyst to produce, at least, one or more dialkyl carbonates and one or more glycols. In at least one embodiment, the one or more dialkyl carbonates may be at least partially removed from the crude product stream of the transesterification reactor system 322 via line 326. Such removal processes may include any suitable one or more processes, such as a series of distillation processes. The remaining crude product stream of the transesterification reactor system 322 may be introduced to a separation apparatus 330 via line 328. In some embodiments, separation apparatus 330 may be equipped with one or more of a distillation separating apparatus, an extractive distillation separating apparatus, a liquid-liquid extraction separating apparatus, a crystallization separating apparatus, an absorption separating apparatus, and/or a membrane separating apparatus so as to obtain a mono-glycolic and/or dialkyl carbonate products from the product stream of the epoxidation reaction (line 332).

In some embodiments, the processes, parameters, and/or reactants of the epoxidation reactor system 304 may include any such processes, parameters, and/or reactants previously disclosed, such as those disclosed for the epoxidation reaction of reactor system 104. In some embodiments, the processes, parameters, and/or reactants of the reaction reactor system 318 used to form cyclic carbonates from the reaction of one or more alkylene oxides with CO₂ may include any such processes, parameters, and/or reactants previously disclosed, such as those disclosed for the reactor system 110. In some embodiments, the processes, parameters, and/or reactants of the transesterification reactor system 322 may include any such processes, parameters, and/or reactants previously disclosed, such as those disclosed for the transesterification reactor system 114. In some embodiments, the processes, parameters, and/or apparatuses of the separation apparatus 330 used to separate components of the crude product stream of the transesterification reactor system 322 may include any such processes, parameters, and/or apparatuses previously disclosed, such as those disclosed for the separation apparatus 122.

### Water Content and Effects Thereof

In some embodiments, a method for removing water and other contaminants from the crude product stream of the epoxidation reaction (e.g., reactor system 304) may include quenching the crude product stream with a cooled/chilled recirculating aqueous solution. In one or more embodiments, the temperature of the crude product stream of the epoxidation reaction is lowered such that the temperature will reduce the water content in the gas feed. In at least one embodiment, a portion of the recirculating aqueous solution may be withdrawn as a bleed stream from the quench section, and any alkylene oxide in the bleed stream may be recovered by any suitable method, such as distillation. In at least one embodiment, basic compounds (e.g., sodium hydroxide) may be added to the recirculating aqueous solution to enhance removal of contaminants. In some embodiments, a method for removing water and/or other contaminants may include cooling the gas stream using heat exchangers, causing condensation of water that can thereafter be removed.

Interestingly, it has been found that processes disclosed herein for the simultaneous formation of dialkyl carbonates and mono-glycolic compounds exhibit a tolerance to water and other reaction side-products/contaminants. For example, it has been found that aspects pertaining to the formation of cyclic carbonates via the reaction between an alkylene oxide and CO₂ can be improved in the presence of at least a percentage of water. In some embodiments, the conversion rate of the reaction between alkylene oxides and CO₂ to form cyclic carbonates increases when in the presence of at least a percentage of water. Such an increase in the conversion rate to cyclic carbonates comes at the expense reaction selectivity, wherein increasing water content not only increases conversion rate in the formation of cyclic carbonates but also increases the amount of glycol formation. However, such processes for the formation of cyclic carbonates disclosed herein have been shown to exhibit tolerance to various possible impurities, which could be formed during such reaction processes. As such, crude alkylene oxide streams from processes disclosed herein may be considered as an easily integrated feed source for the formation of cyclic carbonate compounds.

In continuous and/or sequential processes disclosed herein, the transesterification reaction may also benefit from a balance in water content present in the crude reactant stream. As previously noted, increasing water content in the crude product stream of the epoxidation reaction increases conversion rate in the formation of cyclic carbonates at the expense of reaction selectivity (e.g., glycol formation). However, the loss in selectivity can be considered a benefit for the transesterification reaction as water is a strong poison for such reaction processes, whereas glycol has been shown to have no effect. As such, it has been shown that forming glycol as a result of hydrolysis during the formation of the cyclic carbonate only results in no overall yield loss or only a small overall yield loss of the alkylene carbonate to be produced in subsequent transesterification reactions when crude feed streams are used.

### Additional Aspects

Embodiments disclosed herein include:
A: A process comprising: (a) epoxidizing an olefin in the presence of a first catalyst to produce a first product stream comprising an alkylene oxide; (b) reacting at least a portion of the first product stream with carbon dioxide in the presence of a second catalyst to produce a second product stream comprising a cyclic carbonate, wherein the second product stream has a water content less than about 0.5 wt % based on the second product stream; and (c) reacting at least a portion of the second product stream with an alcohol in the presence of a third catalyst to produce a third product stream comprising aa dialkyl carbonate.
B: A process includes providing a first composition comprising an alkylene oxide and at least one of: water and glycol, wherein the first composition has a water content of less than about 5 wt % based on the first composition and a glycol content less than about 5 wt % based on the first composition; and introducing carbon dioxide to the first composition in the presence of a catalyst to produce a second composition comprising a cyclic carbonate and a water content less than about 0.5 wt % based on the second composition, wherein the second composition comprises the catalyst in an amount of about 0.1 wt % to about 5 wt % based on the second composition, wherein producing the second composition comprises converting about 80% or greater of the alkylene oxide to a combination of glycol and the cyclic carbonate.
C: A process includes providing a first composition comprising an alkylene oxide, and at least one of: water and glycol, wherein the first composition has a water content of less than about 5 wt % based on the first composition and a glycol content less than about 5 wt % based on the first composition, carbon dioxide, and a first catalyst to a first reactor to produce a second composition comprising a cyclic carbonate, wherein an initial molar ratio of carbon dioxide to alkylene oxide in the first composition is from about 25:1 to about 1:1; and introducing the second composition, an alcohol, and a second catalyst to a second reactor to produce a third composition comprising a dialkyl carbonate.

Embodiment A may have one or more of the following additional elements:

Element 1: wherein the olefin is selected from the group consisting of ethylene, propylene, butene, 1-octene, butadiene, allyl chloride, allyl alcohol, styrene, and combinations thereof. Element 2: wherein the first product stream additionally comprises a water content of about 0.05 wt % to about 2.5 wt % based on the first product stream. Element 3: wherein the first catalyst is selected from the group consisting of: a solubilized molybdenum catalyst, a heterogeneous titania on silica catalyst, a silver catalyst, a titanium silicate catalyst, and a noble metal-modified titanium zeolite. Element 4: wherein the second catalyst is selected from the group consisting of tetraethyl ammonium bromide (TEAB), potassium iodide, and a combination thereof. Element 5: wherein the alcohol comprises one or more aliphatic monohydric alcohols. Element 6: wherein the alcohol is methanol. Element 7: wherein the dialkyl carbonate has a Cl symmetry. Element 8: wherein the dialkyl carbonate is dimethyl carbonate. Element 9: wherein the third catalyst is selected from the group consisting of potassium methoxide, sodium methoxide, diazabicycloundecene (DBU), diazabicyclononene (DBN), 1,1,3,3-tetramethyl guanidine, and a combination thereof. Element 10: wherein the first product stream further comprises a glycolic compound. Element 11: wherein the glycolic compound in the first product stream comprises monopropylene glycol (MPG). Element 12: wherein the second product stream further comprises a glycolic compound. Element 13: wherein the glycolic compound in the first product stream comprises monoethylene glycol (MEG). Element 14: additionally comprising separating at least a portion of the dialkyl carbonate from the third product stream. Element 15: wherein separating at least a portion of the dialkyl carbonate from the third product stream is performed in one or more of a distillation separating apparatus, an extractive distillation separating apparatus, a liquid-liquid extraction separating apparatus, a crystallization separating apparatus, an absorption separating apparatus, and a membrane separating apparatus. Element 16: additionally comprising separating at least a portion of the glycolic compound from the third product stream. Element 17: wherein at least a portion of the glycolic compound produced by reacting at least a portion of the second product stream is recycled into the first product stream. Element 18: wherein the first product stream comprises a water content of about 0.01 wt % to about 5 wt %. Element 19: wherein the glycolic compound in the second product stream comprises monopropylene glycol (MPG). Element 20: wherein the glycolic compound in the second product stream comprises monoethylene glycol (MEG).

Each of embodiments B and C may have one or more of the following additional elements:

Element 21: wherein the catalyst is selected from the group consisting of tetraethyl ammonium bromide (TEAB), potassium iodide, or a combination thereof. Element 22: wherein the glycol is monopropylene glycol (MPG). Element 23: wherein the MPG represents less than 1 wt % of the second composition. Element 24: wherein the first catalyst is present in the first composition in an amount of about 0.01 wt % to about 5 wt %. Element 25: wherein the first composition comprises an initial molar ratio of carbon dioxide to propylene oxide of about 4:1 to about 1:1. Element 26: wherein producing the first composition comprises maintaining a temperature of about 100 °C to about 225 °C within the first reactor. Element 27: wherein producing the first composition comprises maintaining a pressure of about 300 psig to about 3,000 psig within the first reactor. Element 28: wherein producing the second composition comprises converting about 50% or greater of the alkylene oxide to a combination of glycol and the cyclic carbonate. Element 29: wherein producing the second composition comprises converting the alkylene oxide to about 80% or greater cyclic carbonate. Element 30: wherein producing the second composition comprises converting about 20% or less of the alkylene oxide to glycol. Element 31: wherein the alcohol is selected from: methanol, ethanol, and combinations thereof. Element 32: wherein the second catalyst is initially present in the second composition in a weight ratio of catalyst to cyclic carbonate of about 0.0005:1 to about 0.05:1. Element 33: wherein the second composition comprises an initial molar ratio of alcohol to cyclic carbonate of about 2:1 to about 8:1. Element 34: wherein the second composition comprises a water content of less than about 0.5 wt %. Element 35: wherein producing the third composition comprises maintaining a temperature of about 50 °C to about 250 °C within the second reactor. Element 36: wherein producing the third composition comprises maintaining a pressure of about 20 psig to about 300 psig within the second reactor. Element 37: wherein producing the third composition comprises converting about 50% or greater of the cyclic carbonate to a combination of glycol and the dialkyl carbonate. Element 38: wherein the third composition comprises about 1 mol % to about 25 mol % dialkyl carbonate. Element 39: wherein the first catalyst is selected from the group consisting of tetraethyl ammonium bromide (TEAB), potassium iodide, and a combination thereof. Element 40: wherein the dialkyl carbonate is selected from the group consisting of: dimethyl carbonate, diethyl carbonate, ethylmethyl carbonate, or a combination thereof. Element 41: wherein the second catalyst is selected from the group consisting of potassium methoxide, sodium methoxide, diazabicycloundecene (DBU), diazabicyclononene (DBN), 1,1,3,3-tetramethyl guanidine, and a combination thereof.

### EXAMPLES

### Example 1

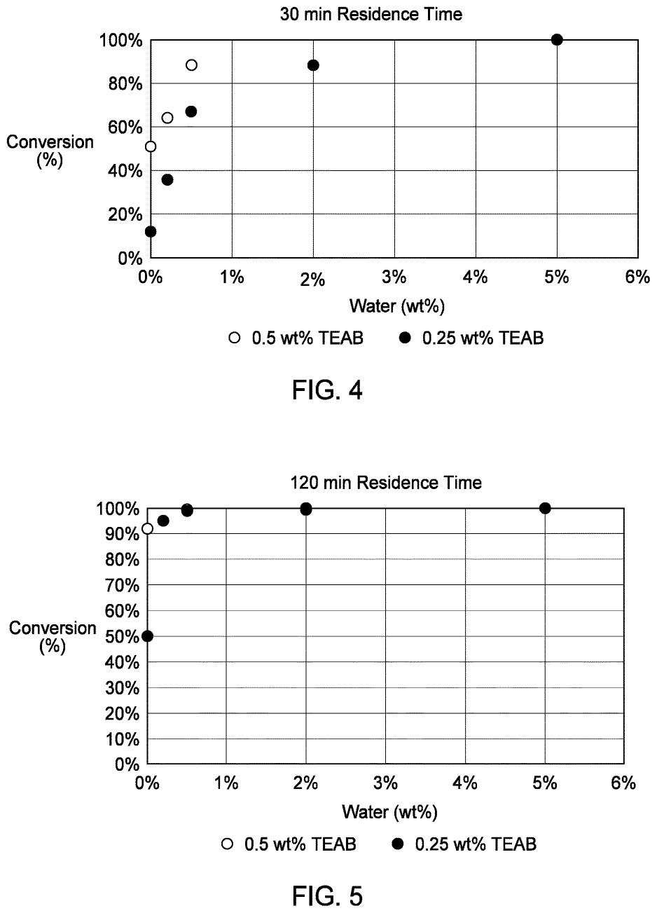

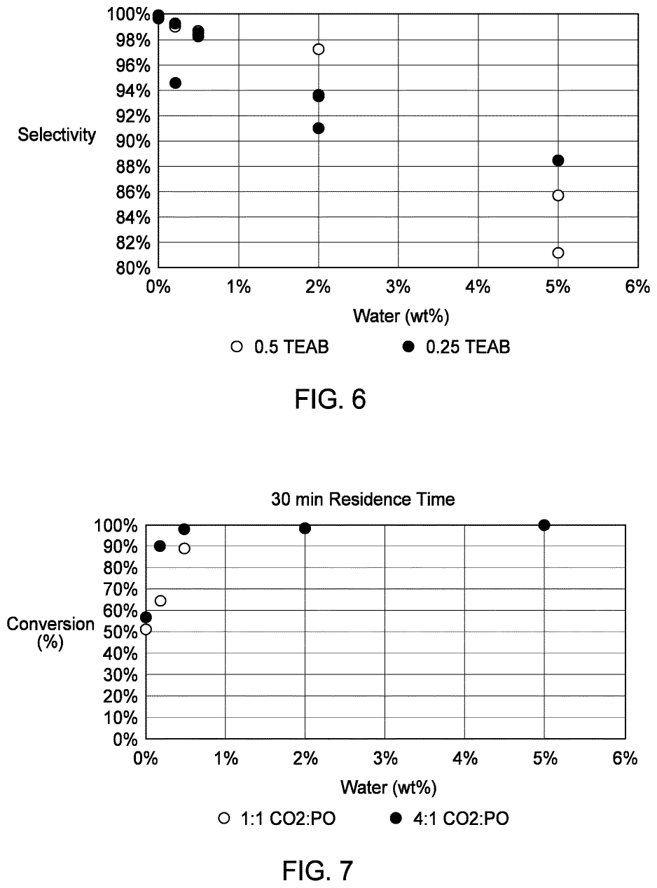

A series of reactions were conducted wherein propylene oxide was reacted with CO₂ in the presence of water to produce propylene carbonate. Each of the reactions were conducted using the same temperature (170 °C), pressure (700 psig), catalyst identity (tetraethylammonium bromide; hereinafter referred to as “TEAB”), and a molar ratio of CO₂ to epoxide of 1:1. The various other variables were altered to determine the selectivity and conversion efficiency of such reactions. For instance, the water content present in the reactions were 0 wt %, 0.25 wt %, 0.5 wt %, 2 wt %, and 5 wt % of the total reaction composition. Additionally, the catalyst content (TEAB) for each of the different water contents were 0.25 wt % and 0.5 wt %. The reaction times for each of the different water contents were 30 minutes and 120 minutes. Reaction conversion was monitored for each of the reaction conditions and recorded as a function of water content. The conversion results are illustrated in FIG. 4 and FIG. 5. Additionally, the selectivity of such reactions was determined by monitoring the amount of propylene carbonate and propylene glycol produced for each of the reaction conditions using a reaction time of 120 minutes, wherein the percentage of propylene carbonate present in the product stream was recorded as a function water content. The selectivity results are illustrated in FIG. 6. As is evidenced by FIG. 4-6, increasing water content increases reaction rate. However, the increase in reaction rate comes at the detriment of reaction selectivity.

### Example 2

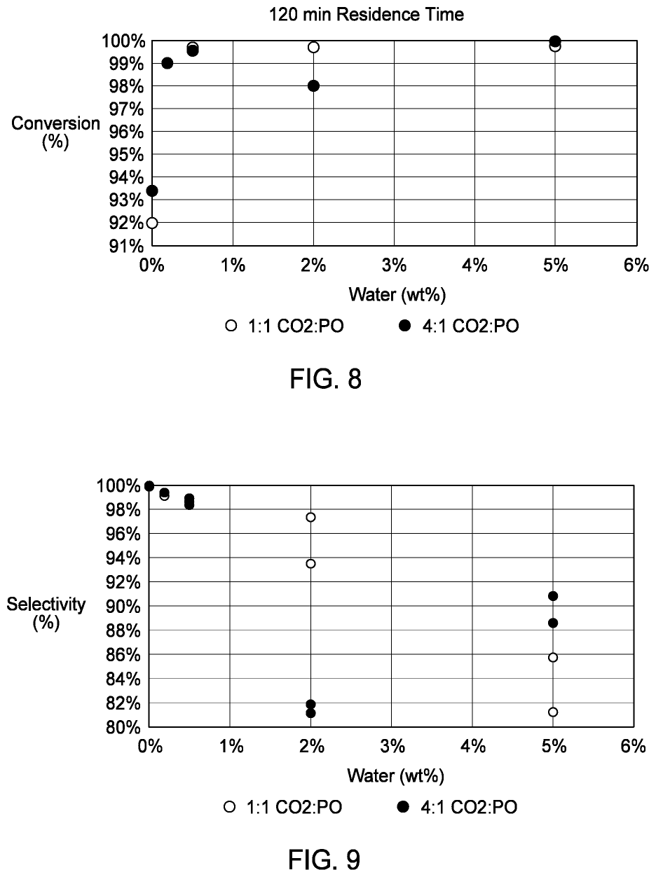

A series of reactions were conducted wherein propylene oxide was reacted with CO₂ in the presence of water to produce propylene carbonate. Each of the reactions were conducted using the same temperature (170 °C), pressure (700 psig), catalyst identity (TEAB), and a catalyst content of 0.25 wt %. The various other variables were altered to determine the selectivity and conversion efficiency of such reactions. For instance, the water content present in the reactions were 0 wt %, 0.25 wt %, 0.5 wt %, 2 wt %, and 5 wt % of the total reaction composition. Additionally, the CO₂ to propylene oxide ratio present in the reaction mixture was 1:1 and 4:1. The reaction times for each of the different water contents were 30 minutes and 120 minutes. Reaction conversion was monitored for each of the reaction conditions and recorded as a function of water content. The conversion results are illustrated in FIG. 7 and FIG. 8. Additionally, the selectivity of such reactions was determined by monitoring the amount of propylene carbonate and propylene glycol produced for each of the reaction conditions using a reaction time of 120 minutes, wherein the percentage of propylene carbonate present in the product stream was recorded as a function water content. The selectivity results are illustrated in FIG. 9. As is evidenced by FIG. 7-9, increasing the ratio of CO₂ to propylene oxide in the reaction mixture results in slight increases in both reaction rate and reaction selectivity.

### Example 3

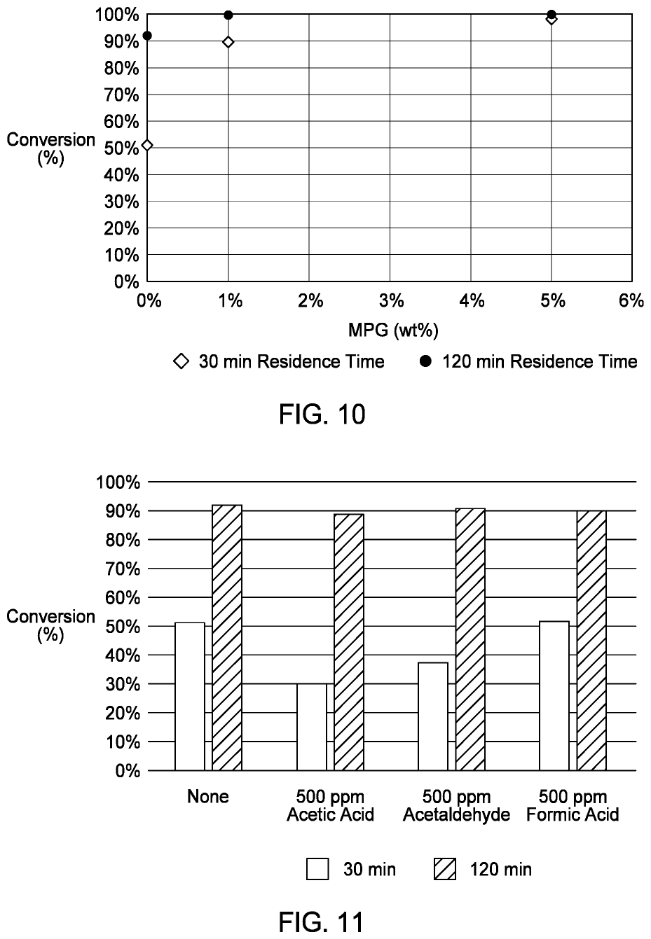

A series of reactions were conducted wherein propylene oxide was reacted with CO₂ in the presence of propylene glycol to produce propylene carbonate. Each of the reactions were conducted using the same temperature (170 °C), pressure (700 psig), catalyst identity (TEAB), a catalyst concentration of about 0.5 wt %, and a molar ratio of CO₂ to epoxide of 1:1. The other variables were altered to determine the selectivity and conversion efficiency of such reactions. For instance, the propylene glycol content present in the reactions were 0 wt %, 1 wt %, and 5 wt % of the total reaction composition. The reaction times for each of the different water contents were 30 minutes and 120 minutes. Reaction conversion was monitored for each of the reaction conditions and recorded as a function of propylene glycol content. The conversion results are illustrated in FIG. 10. As is evidenced by FIG. 10, having propylene glycol present in the reaction composition results in a similar increase in reaction rate as compared to those having water content present (e.g., FIG. 4-5). However, the increase is not as pronounced.

### Example 4

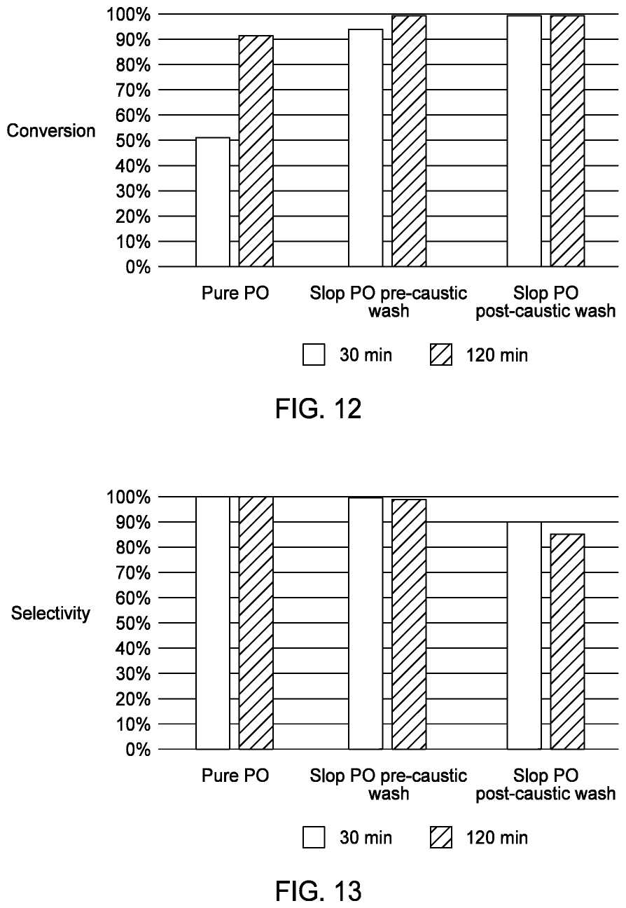

A series of reactions were conducted wherein propylene oxide was reacted with CO₂ produce propylene carbonate. Each of the reactions were conducted using the same temperature (170 °C), pressure (700 psig), catalyst identity (TEAB), a catalyst concentration of about 0.5 wt %, and a molar ratio of CO₂ to epoxide of 1:1. The reactions were conducted in the presence of known impurities (e.g., 500 ppm of acetic acid, acetaldehyde, or formic acid) of crude product streams of epoxidation reactions to determine the tolerability of the reaction to such components. Reaction conversion was monitored for each of the reactions, and the results are illustrated in FIG. 11. Additionally, reactions were conducted using pure propylene oxide, pre-treated crude propylene oxide (also referred to as slop) propylene oxide, and post-treated slop propylene oxide streams. Reaction conversion and selectivity was monitored for each of the reactions, and the results are illustrated in FIG. 12-13. As evidenced by FIG. 11-13, the processes, as disclosed herein, of forming propylene carbonate exhibit tolerability to potential impurities, which may be present in crude product epoxidation streams. In fact, the illustrated in FIG. 12 suggest that using crude propylene oxide streams results in higher conversion rates than reactions using pure propylene oxide. Thus, it may not be necessary to expend time and economic resources to purify such feed streams.

### Example 5

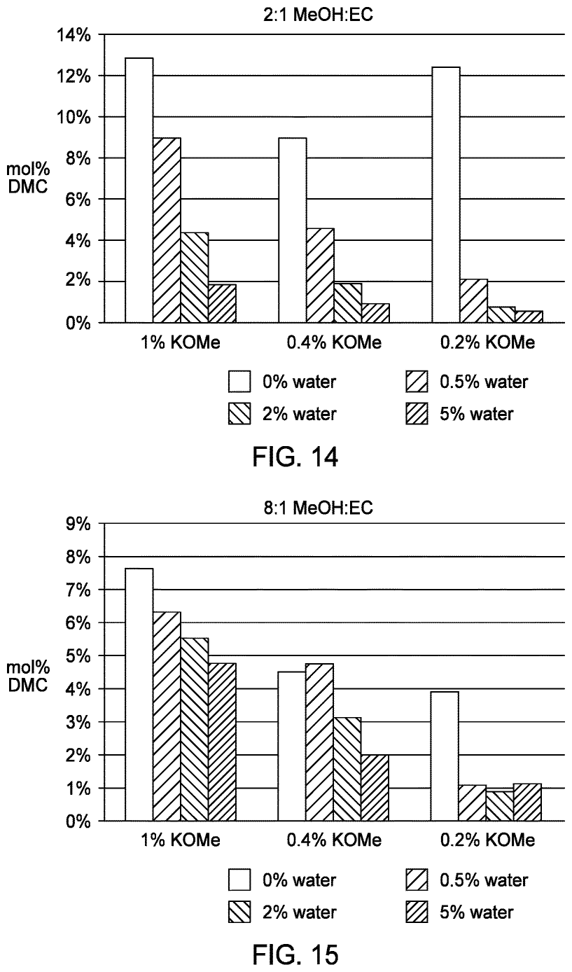

A series of transesterification reactions were conducted wherein ethylene carbonate was reacted with methanol to produce dimethyl carbonate and ethylene glycol. Each of the transesterification reactions were conducted using the same temperature (50 °C), pressure (600 psig), catalyst identity (potassium methoxide, hereinafter referred to as KOMe), and reaction time (1 minute). The various other variables were altered to determine the conversion efficiency of such reactions in terms of the production of dimethyl carbonate. For instance, the water content present in the reactions were 0 vol %, 0.5 vol %, 2 vol %, and 5 vol % of the total reaction composition. Additionally, the catalyst content (KOMe) for each of the different water contents were 1 mol %, 0.4 mol %, and 0.2 mol %. Furthermore, the molar ratio of methanol to ethylene carbonate for each of the reactions was 2:1 and 8:1. Reaction conversion was monitored for each of the reaction in terms of the molar amount of dimethyl carbonate formed therein. The results are illustrated in FIG. 14-15. As evidenced by FIG. 14-15, increased water content present in conjunction with lower molar ratios of methanol to ethylene carbonate result in a higher inhibition of the reaction. Additionally, the results illustrated in FIG. 15 illustrate that increased catalyst content (KOMe) results in increased dimethyl carbonate formation.

### Example 6

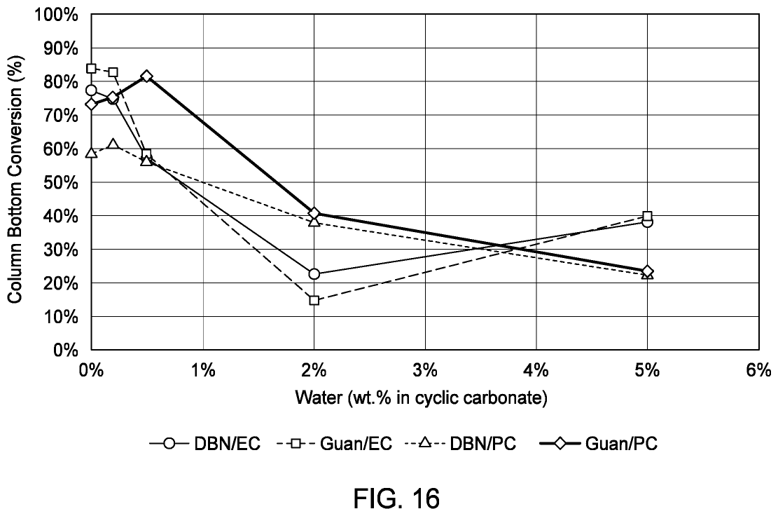

A series of transesterification reactions were conducted wherein ethylene carbonate was reacted with methanol to produce dimethyl carbonate and ethylene/propylene glycol. Each of the transesterification reactions was conducted using the same temperature (50 °C), pressure (600 psig), catalyst concentration (4 mol %), and reaction time (1 minute). The water content (e.g., 0 vol %, 0.5 vol %, 2 vol %, and 5 vol %) and catalyst identity (e.g., amidine and/or guanidine catalysts) were altered to determine the conversion efficiency of such reactions in terms of the production of dimethyl carbonate when implementing an 8:1 molar ratio of methanol to cyclic carbonate. The results are illustrated in FIG. 16. It was found that water inhibits dimethyl carbonate formation when such catalyst compositions are integrated within the reaction. Such water inhibition becomes substantial at a water content of about 0.5 wt %.

### Example 7

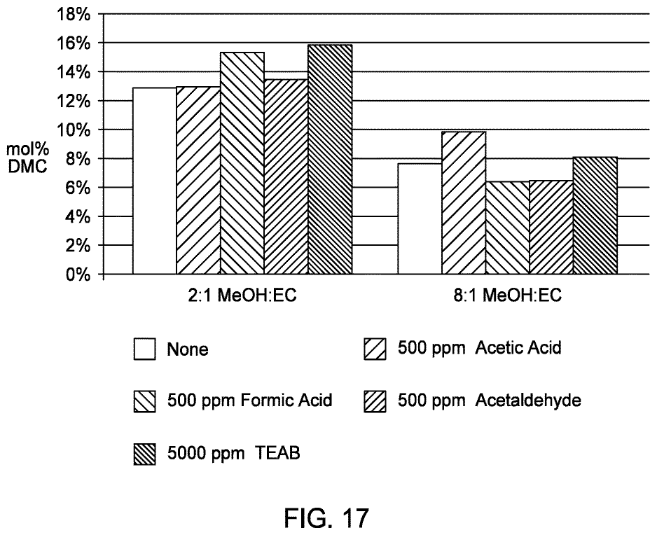

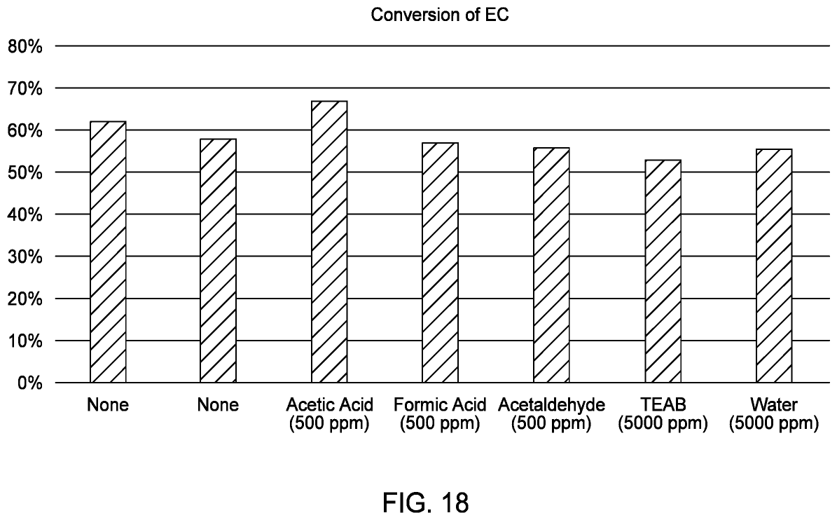

A series of transesterification reactions were conducted wherein ethylene carbonate was reacted with methanol to produce dimethyl carbonate and ethylene glycol. Each of the transesterification reactions were conducted using the same temperature (50 °C), pressure (600 psig), catalyst identity (KOMe), catalyst content (1 mol %), and reaction time (1 minute). The reaction were conducted in the presence of various potential impurities (e.g., 500 ppm acetic acid, 500 ppm formic acid, 500 ppm acetaldehyde, or 5000 ppm TEAB) to determine the tolerability of the reaction to such components. Reaction conversion was monitored for each of the reactions, and the results are illustrated in FIG. 17. Reaction conversion was monitored for each of the reactions in terms of the molar amount of dimethyl carbonate formed therein, and the results are illustrated in FIG. 17. As evidenced by FIG. 17-18, the transesterification reaction exhibits high degrees of tolerability to various potential impurities which may be present in a crude product feed stream provided from a cyclic carbonate formation process previously described herein.

### Example 8

A series of reactions were conducted wherein ethylene oxide was reacted with CO₂ produce ethylene carbonate. Each of the reactions were conducted using the same temperature (140 °C), pressure (700 psig), catalyst identity (TEAB), a catalyst concentration of about 0.25 wt %, and a molar ratio of CO₂ to epoxide of 2:1. The reactions were conducted in the presence of known impurities (e.g., 500 ppm of acetic acid, acetaldehyde, or formic acid), added water (e.g. 0.5 wt % or 2 wt %), or ethylene glycol (e.g. 1 wt %) of crude product streams of epoxidation reactions to determine the tolerability of the reaction to such components. Reaction conversion was monitored for each of the reactions, and the results are illustrated in Table 1. As evidenced by Table 1, the processes, as disclosed herein, of forming ethylene carbonate exhibit the same reaction behavior as propylene oxide to potential impurities, which may be present in crude product epoxidation streams. Thus, it may not be necessary to expend time and economic resources to purify such feed streams.

TABLE 1

| Condition | Conversion (%) | Selectivity (%) |
|---|---|---|
| Baseline | 23 | 100 |
| Baseline | 17 | 100 |
| 500 ppm formic and acetic acids | 20 | 100 |
| 500 ppm acetaldehyde | 23 | 100 |
| 1 vol % methyl ethyl glycol (MEG) | 32 | 100 |
| 0.5 vol % water | 41 | 100 |
| 2 vol % water | 40 | 100 |

### Example 9

A series of transesterification reactions were conducted wherein propylene carbonate was reacted with methanol to produce dimethyl carbonate and propylene glycol. Each of the transesterification reactions were conducted using the same temperature (50 °C), pressure (600 psig), catalyst identity (KOMe), catalyst content (1 mol %), and reaction time (1 minute). The reactions were conducted in the presence of various potential impurities (e.g., 500 ppm acetic acid, 500 ppm formic acid, 500 ppm acetaldehyde, or 5000 ppm TEAB), or water (e.g. 0.5 vol %, 5 vol %) to determine the tolerability of the reaction to such components. Reaction conversion was monitored for each of the reactions in terms of the molar amount of dimethyl carbonate formed therein, and the results are illustrated in Table 2 for two different runs-a first having a molar ratio of methanol to propylene carbonate (MeOH:PC) of 1:1 and the second having a molar ratio of methanol to propylene carbonate of 2:1. As evidenced by Table 2, the transesterification reaction exhibits high degrees of tolerability to various potential impurities except water which may be present in a crude product feed stream provided from a cyclic carbonate formation process previously described herein. Increased water content resulted in an increased inhibition of the reaction.

TABLE 2

| | Dimethyl carbonate yield | | | | | | |
|---|---|---|---|---|---|---|---|
| Molar Ratio | | 5 vol % | 0.5 vol % | 500 ppm | 500 ppm | 500 ppm | 0.5 vol % |
| MeOH:PC | None | H₂0 | H₂0 | acetic acid | formic acid | acetaldehyde | TEAB |
| 1:1 | 9.71% | 0.45% | 2.49% | 8.99% | 7.25% | 10.74% | 10.44% |
| 2:1 | 7.25% | 0.51% | 3.29% | 7.30% | — | 7.29% | 9.92% |

Overall, the present disclosure relates to methods of simultaneous formation of dialkyl carbonates and mono-glycolic compounds via the integration of dialkyl carbonate formation processes into commercially implemented pathways of glycolic compound production. The reactions and processes disclosed herein can implement crude product streams of epoxidation reactions in the formation of cyclic carbonates, wherein the crude product streams from the formation of cyclic carbonates are implemented as a feed stream for the transesterification reaction. Separation processes described herein allow for an operator to separate dialkyl carbonates from mono-glycolic compounds formed via the transesterification reaction of the parent cyclic carbonate and aliphatic monohydric alcohols. Additionally, reactions and processes disclosed herein have shown to be tolerable to potential impurities and by-products which may develop during operation. In fact, it has been found that some impurities (e.g., water) may increase reaction rates of such processes. Such reactions and reactant feed streams disclosed herein exemplify the tolerability and economic feasibility of integrating such dialkyl formation processes into commercially implemented pathways of glycol production.

The phrases, unless otherwise specified, “consists essentially of” and “consisting essentially of” do not exclude the presence of other steps, elements, or materials, whether or not, specifically mentioned in this specification, so long as such steps, elements, or materials, do not affect the basic and novel characteristics of the present disclosure, additionally, they do not exclude impurities and variances normally associated with the elements and materials used.

Numerical ranges used herein include the numbers recited in the range. For example, the numerical range “from 1 wt % to 10 wt %” includes 1 wt % and 10 wt % within the recited range.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited.

All numerical values within the detailed description herein are modified by “about” the indicated value, and take into account experimental error and variations that would be expected by a person having ordinary skill in the art.

All documents described herein are incorporated by reference herein, including any priority documents and or testing procedures to the extent they are not inconsistent with this text. As is apparent from the foregoing general description and the specific embodiments, while forms of the present disclosure have been illustrated and described, various modifications can be made without departing from the spirit and scope of the present disclosure. Accordingly, it is not intended that the present disclosure be limited thereby. Likewise, the term “comprising” is considered synonymous with the term “including” for purposes of United States law. Likewise whenever a composition, an element or a group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

While the present disclosure has been described with respect to a number of embodiments and examples, those skilled in the art, having benefit of this disclosure, will appreciate that other embodiments can be devised which do not depart from the scope and spirit of the present disclosure.

1. A process comprising:
epoxidizing an olefin in the presence of a first catalyst to produce a first product stream comprising an alkylene oxide;
reacting at least a portion of the first product stream with carbon dioxide in the presence of a second catalyst to produce a second product stream comprising a cyclic carbonate, wherein the second product stream has a water content less than about 0.5 wt % based on the first reaction composition; and
reacting at least a portion of the second product stream with an alcohol in the presence of a third catalyst to produce a third product stream comprising a dialkyl carbonate.

2. The process of claim 1, wherein the olefin is selected from the group consisting of ethylene, propylene, butene, 1-octene, butadiene, allyl chloride, allyl alcohol, styrene, and combinations thereof.

3. The process of claim 1, wherein the first product stream additionally comprises a water content of about 0.05 wt % to about 2.5 wt % based on the first product stream.

4. The process of claim 1, wherein the first catalyst is selected from the group consisting of: a solubilized molybdenum catalyst, a heterogeneous titania on silica catalyst, a silver catalyst, a titanium silicate catalyst, and a noble metal-modified titanium zeolite.

5. The process of claim 1, wherein the second catalyst is selected from the group consisting of tetraethyl ammonium bromide (TEAB), potassium iodide, and a combination thereof.

6. The process of claim 1, wherein the alcohol comprises one or more aliphatic monohydric alcohols.

7. The process of claim 1, wherein the alcohol is methanol.

8. The process of claim 1, wherein the dialkyl carbonate has a Cl symmetry.

9. The process of claim 1, wherein the dialkyl carbonate is dimethyl carbonate.

10. The process of claim 1, wherein the third catalyst is selected from the group consisting of potassium methoxide, sodium methoxide, diazabicycloundecene (DBU), diazabicyclononene (DBN), 1,1,3,3-tetramethyl guanidine, and a combination thereof.

11. The process of claim 1, wherein the first product stream further comprises a glycolic compound.

12. The process of claim 11, wherein the glycolic compound is monopropylene glycol (MPG).

13. The process of claim 1, wherein the second product stream further comprises a glycolic compound.

14. The process of claim 13, wherein the glycolic compound is monoethylene glycol (MEG).

15. The process of claim 13, additionally comprising: separating at least a portion of the dialkyl carbonate from the third product stream.

16. The process of claim 15, wherein separating at least a portion of the dialkyl carbonate from the third product stream is performed in one or more of a distillation separating apparatus, an extractive distillation separating apparatus, a liquid-liquid extraction separating apparatus, a crystallization separating apparatus, an absorption separating apparatus, and a membrane separating apparatus.

17. The process of claim 13, additionally comprising separating at least a portion of the glycolic compound from the third product stream.

18. The process of claim 13, wherein at least a portion of the glycolic compound produced by reacting at least a portion of the second product stream is recycled into the first product stream.

19. The process of claim 1, wherein the first product stream comprises a water content of about 0.01 wt % to about 5 wt %.
